(19) United States
(12) Patent Application Publication
(10) Pub. No.: US 2007/0020209 A1
(43) Pub. Date: Jan. 25, 2007

(54) Makeup compositions and methods

(76) Inventors: Tatyana Zamyatin, Princeton Junction, NJ (US); Ida Marie Sandewicz, Monroe Township, NJ (US); Julio Gans Russ, Westfield, NJ (US); Ivonne Brown, Roosevelt, NY (US); Merry Lee Nickl, Kingston, NJ (US); Andrea Kroenig, Montclair, NJ (US)

Correspondence Address: Julie Blackburn;Revlon Consumer Products Corporation, 237 Park Avenue, New York, NY, 10017, US

(21) Appl. No.: 11/384,138

(22) Filed: Mar. 17, 2006

Related U.S. Application Data
(60) Provisional application No. 60/700,969, filed on Jul. 20, 2005.

(51) Int. Cl. A61K 8/73; A61K 8/19

(52) U.S. Cl. 424/63; 424/70.13

(57) ABSTRACT

Emulsion makeup compositions for keratinous surfaces which change color upon application, emulsion makeup compositions that match a variety of skin or hair shades in more than one skin tone category, and methods for treating keratinous surfaces with an emulsion cosmetic composition that changes color upon application.

### RELATED APPLICATIONS

This application claims priority from provisional patent application ser. no. 60/700,969, filed Jul. 20, 2005.

### TECHNICAL FIELD

The invention is in the field of emulsion makeup compositions for application to keratinous surfaces that change color when applied to the keratinous surface. In one embodiment, the emulsion compositions are for application to skin, and match a plurality of skin shades and develop color when applied to the skin. The invention is also directed to a method for formulating cosmetic compositions for application to keratinous surfaces where the color of the composition changes upon application to the keratinous surface. The invention is also directed to a method for formulating cosmetic compositions that are suitable for a wide variety of skin or hair shades so as to eliminate the number of SKUS cosmetics manufacturers must stock, as well as to reduce the resulting consumer confusion from having too many options to choose from.

### BACKGROUND OF THE INVENTION

Color matching is a common concern with respect to color cosmetics that are applied to keratinous surfaces such as skin, lips, or eyelashes and eyebrows. With respect to foundation makeup in particular, in order to ensure that there is a suitable shade for every consumer skin color, cosmetics companies will often offer up to 30 shades in a particular brand so that all possible skin colors are represented. One common problem for both consumers that purchase foundation makeup and the cosmetics companies that sell such products, is to ensure that the consumer purchases the color that best matches their skin color. This is a particular issue when consumers purchase foundation from outlets in the self-select channel of distribution. The term “self-select” means, with respect to the outlet in which the consumer is shopping, that the consumer selects the products by herself generally without the advice and counsel of a beauty advisor. Typically self-select channels include large mass market retailers such as KMart, WalMart, or Target; or chain drug stores such as CVS, Rite-Aid, or Walgreen's. In these shopping situations, the consumer must select the proper shade under conditions, such as fluorescent lighting, that may not contribute to purchase of the product that is best matched with the skin color.

Similarly, for color cosmetics such as blush, eye shadow, and the like, the consumer has almost too many colors to choose from. Having too many options to select from is almost as problematic for consumers as having too few options. To simplify the shopping experience for these types of color cosmetics, it is desirable to have only a few options that match all, or nearly all keratinous surfaces.

Cosmetics companies that have a substantial mass market business have tried to address the matching problem in a variety of ways. For example, L'Oréal sells a line of foundation makeup, powder, concealer and blush products under the True Match® brand. Selection of the foundation that best matches the consumer's skin color is performed in two steps. First, the shopper is asked to select the color chip that is closest to her skin color from a palette offering 10 possible choices (such as fair, light, light/medium, medium, etc). In the second step, the shopper is asked to select her skin undertone from 3 possible choices (warm, neutral, cool). The cosmetics display then indicates which of the approximately twenty four different shades are most suitable for that particular consumer. While this method of selecting foundation to ensure a true match exhibits certain improvements over the traditional method of selling foundation, it still requires that the consumer be capable of making interpretations that require some skill. Not all consumers are sure of what their skin color is, especially when there are ten possible color chips to choose from. This decision is also made more difficult under the fluorescent lighting conditions that are often found in stores. Further complicating the decision process, is requiring the consumer to then determine skin undertone. The possibility for error in the consumer's interpretation is reasonably high, with the end result being the purchase of a foundation product that does not truly match the purchaser's skin color. With respect to the blush, twelve different shades are available; nearly as many as found in standard cosmetics lines. Thus, considerable consumer interpretation is still necessary.

Procter & Gamble's Cover Girl brand uses a similar approach to solve the foundation matching problem with their product line Tru Blend®. The self-select shopper is asked to determine her skin tone from a palette of about 10 different shades; then determine her skin undertone (generally cool, neutral, or warm). The foundation suitable for the shopper is then identified based upon the consumer's interpretation of her skin color and undertone, and the decisions made with respect to the choices offered. The margin for error is the same as with the L'Oréal products. While consumers tend to make better color matches using the Tru Blend® system, there is still a significant margin for error.

Accordingly, there is a need for simplifying the shopping experience for the consumer to ensure that the products suitable for that consumer are easily identifiable by the consumer herself, without requiring skilled interpretation or decision making. One general way to accomplish this is to reduce the number of choices the consumer is obliged to make in selecting a suitable foundation makeup, blush, eyeshadow, concealer, mascara, brow color or other color products, and make the consumer interpretation required as simple as possible. For example, one obvious way to do this, is to provide three or four general categories and ask the consumer to determine what category she falls into. For example, such skin tone categories might be “light”, “light/medium”, “medium”; or “dark”. Or such eyelash or eyebrow categories may be “light”, “brown”, “black”. Once the consumer had made that one decision, there would be one foundation makeup suitable for all skin shades in the one skin tone category selected; or a blush suitable for all, or more than one skin shade in one skin tone category; or a mascara suitable for all lash colors or which provides a unique second effect upon application. In the case of foundation makeup, the product would be suitable on all skin shades generally found within the “light” category (e.g. foundation colors typically described by descriptors such as fair, ivory, buff, nude, etc.). Similarly, for products such as blush, there might be one blush suitable for use with all skin shades in one skin tone category, or possibly one blush suitable for all skin shades across all skin tone categories. In the case where the composition is a concealer or lipstick, there may be one concealer that matches all skin shades in one skin tone category. In the case where the composition is an eyeshadow, there may be several basic eyeshadow colors that are suitable for skin shades in one, or more than one, skin tone category. In the case where the product is a mascara or brow color, one color may be suitable for all different types of lash colors.

Alternatively, in each case, the product may exhibit one color in the resting state and a second color when applied to the desired keratinous surface. Then it is possible to provide a variety of unique visual effects. For example, a mascara composition could be white or light gray in the container, but when applied would turn a suitable lash matching color such as black or brown. Alternatively, the mascara could be a suitable lash matching color such as black or brown in the resting state, but when applied to lashes a second color such as metallic, other color tints, etc. would form on the lashes.

When considering color cosmetics applied to skin such as foundation makeup, the formulation of make ups that are suitable for a wider variety of skin shades requires some skill. For example, it is important that foundation makeup match the skin tone as closely as possible for obvious reasons. Generally, there are at least two factors that contribute to proper matching of skin tone. First is pigmenting. Mixtures of pigments will provide the basic color of the foundation. Typically this coloration is achieved by a blend of iron oxides such as black, yellow, and red. While pigments are responsible for providing the characteristic color to the foundation, they generally do not provide coverage. The term “coverage” means the ability of the foundation to cover or conceal skin imperfections, or otherwise provide a film on the skin. Coverage is generally achieved by including ingredients in the foundation formula that provide opacity. In most cases titanium dioxide or other similar powders will provide the opacity as well as to mute the effect of the pigments that are present. In general, foundations that have high opacity (e.g. powder content) provide greater coverage.

Alternatively, foundations that have lower opacity provide less coverage and a sheerer finish. While increasing the opacity of foundation provides certain benefits with respect to covering skin imperfections, foundations with high opacity are harder to match with skin. When foundation makeup manufacturers sell formulas having higher opacity, they must include many more SKUS in their lines. For example, a foundation manufacturer that sells a relatively high opacity foundation may need to have 24 to 30 shades in the foundation makeup line to ensure that all the skin colors of their customer base are adequately represented. More SKUs means more expense for the cosmetics manufacturer and the retailer and increases consumer confusion about how to select the proper foundation for her skin from the myriad of choices. The same is generally true for color cosmetic products such as blush, eyeshadow, concealer, bronzers, and the like. The greater the opacity in these products the more shades are necessary to provide consumers with a full palette of colors to choose from.

There is clearly a need for color cosmetic products that are suitable for use on a wide variety of skin tones in one or more shade categories. This enables cosmetic manufacturers to reduce the number of SKUs offered for sale in their line. This will minimize cost for the manufacturer, reduce the inventory retailers must maintain, and minimize consumer confusion due to having too many options to select from.

Even more desirable with respect to color cosmetics such as foundation makeup, blush, concealer, eyeshadow, bronzer, lipstick, eyeliner, brow color, mascara, and the like, is a product that matches a variety of skin tones or hair colors, wherein such a composition would exhibit one standard resting color and a second application color so that there is a consumer perception that the cosmetic composition is “smart”, e.g. it changes color to exactly match her skin tone or hair color. The color of such “smart” cosmetic would develop upon application to the keratinous surface, giving the consumer the impression that the makeup changes color from an initial resting color to a color that exactly matches her skin tone or lash color.

It is an object of the invention to provide a color cosmetic composition that is more universal in its ability to match a variety of skin tones or lash or brow colors; and particularly with respect to skin tones, match more in one general category such as “light”, “light/medium”, “medium” or “dark”.

It is a further object of the invention to provide a color cosmetic composition such as foundation makeup, blush, concealer, eyeliner, eyeshadow, brow color, lipstick, mascara, and so on, where the composition enables cosmetics manufacturers to offer products that match the skin, lash, brow, or hair color variations of their customers, but with a reduced number of SKUs.

It is a further object of the invention to provide a foundation makeup, blush, concealer, lipstick, eyeshadow or eyeliner that has a first resting color and a second application color, such that when the makeup is in a resting state (such as in the container) it exhibits one color which may be a very generic color (such as white, grayish white, or bluish white) or a regular color, and upon application the color develops to match the skin tone of the consumer.

It is a further object of the invention to provide a makeup that matches a plurality of skin shades in one skin tone category in order to reduce the number of SKUs sold by cosmetics manufacturers and the consumer confusion involved in trying to select the appropriate foundation color from a multiplicity of shade offerings.

It is a further object of the invention to provide a foundation makeup that is suitable for the multiplicity of skin shades in each general skin tone category.

It is a further object of the invention to provide a color cosmetic product such as blush, concealer, eye shadow and the like, which is suitable for a multiplicity of skin shades in one skin tone category or across more than one skin tone category.

It is a further object of the invention to provide a color cosmetic product such as blush, concealer, eyeshadow and the like, which exhibits a first resting color and a second application color where the first and second color are visually distinctive.

It is a further object of the invention to provide a cosmetic composition for application to lashes, brows, or hair that has a first resting color and a second application color.

It has been discovered that using certain types of pigments in a cosmetic composition enables color change from one color in the resting state to another color upon application. Further, use of such pigments in combination with certain types of particulates having a certain particle size contributes to the composition's ability to be suitable for more than one skin shade in one or more skin tone categories.

### DETAILED DESCRIPTION

I. The Composition

A. Definitions

1. All percentages mentioned herein are percentages by weight unless otherwise indicated.

2. “Diffusive” means, with respect to the pigment, that it is capable of diffusing from one phase of the composition to another phase of the composition or onto the keratinous surface when the composition is applied to the keratinous surface (such as skin, lashes, or hair). By way of example, when a diffusive water soluble or dispersible pigment is suspended in the oil phase of the emulsion it will be capable of diffusing out of the oil phase (for example, into the water phase), or onto the keratinous surface when the emulsion composition is applied to the skin, lashes, or hair. Preferably, this diffusion from one phase to the other will contribute to color development or change. Also contributing to the color change may be the mechanical agitation involved in application of the cosmetic to the keratinous surface, or the temperature or pH change involved when the composition is removed from the container and applied to the desired keratinous surface.

3. “First resting color” means, with respect to the composition, that when it is in the resting state, e.g. stored in a container, it exhibits one visually distinct color.

4. “Second application color” means, with respect to the composition, that when it is applied to the skin (e.g. removed from the resting state), it exhibits a second visually distinct color that is different from the first visually distinctive color.

5. “Visually distinct” means, with respect to color differences, that they can be observed with the naked eye.

6. “Volatile” means that the ingredient has a vapor pressure of greater than about 2 millimeters of mercury at 20° C.

7. “Near volatile” means that the ingredient has a vapor pressure ranging from about 1 to 2 millimeters of mercury at 20° C.

8. “Non-volatile” means that the ingredient has a vapor pressure of less than about 1 millimeter of mercury at 20° C.

9. “SKU” means stock keeping unit, a term generally used by retailers to identify the lowest level of product detail. By way of example, a foundation makeup in the color nude, sold by a cosmetics manufacturer under the brand XY would typically have a unique numeric identifier in the form xxxx-xx, where the first four digit number would indicate foundation brand XY and would be the same across all shades of brand XY, and the second two digit number after the dash would be specific to each separate shade in the brand.

10. “Skin tone” means the general color of the skin, for example, “light”, “light/medium”, “medium”, or “dark”.

11. “Skin Shade” is a particular color within a skin tone category. For example, consumers who have the skin tone “light” include those that have fair, fair/light, or light skin and typically wear foundation shades such as ivory, buff, or nude. Consumers who have the skin tone “light/medium” have a skin color that is a cross between medium and light color and includes those that wear foundation shades such as sand beige, natural beige, or medium beige. Consumers who fall into the skin tone category of “medium” include those that have medium colored skin and typically wear foundation shades such as honey beige, sand beige, warm or golden beige, or early tan. Consumer who fall into the general skin tone category of “deep” have dark skin and generally wear foundation shades such as mocha, caramel, etc. In general, skin shade is a subset of skin tone, and each skin tone category will include more than one skin shade.

12. The term “powder component” refers to the portion of the composition that is made up of powder ingredients when the total amount of all of the powders (such as nylon, titanium dioxide, boron nitride, mica, and so on) are added together.

13. The term “pigment component” refers to the portion of the composition that is made up of pigments when the total amount of all of the pigments (such as red iron oxide, yellow iron oxide, or black iron oxide) in the composition are added together.

B. Water

Preferably, the composition is in the form of an emulsion containing water and/or one or more non-aqueous solvents. The composition may be a water-in-oil or oil-in-water emulsion. Preferably, the composition comprises from about 0.1-99%, preferably from about 0.5-85%, more preferably from about 1-80% by weight of the total composition of water and from about 0.1-99%, preferably from about 0.5-85%, more preferably from about 1-80% by weight of the total composition of oil.

C. Oils

The composition of the invention preferably contains one or more oils. The term “oil’ means an ingredient that is a pourable liquid at room temperature. Suitable oils may be organic, or silicone based, and suggested ranges are from about 0.1-95%, preferably from about 0.5-85%, more preferably from about 1-75% by weight of the total composition.

1. Silicone Oils

Suitable silicone oils may be volatile or non-volatile.

(a). Volatile Silicone Oils

Volatile silicones that may be used in the composition are linear or cyclic. Suitable cyclic volatile silicones have the general formula: 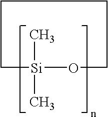
wherein n=3-6.

Examples of such cyclic volatile silicones include hexamethylcyclodisiloxane, octamethylcyclotetrasiloxane, decamethylcyclopentasiloxane, dodecamethylcyclohexasiloxane, and so on.

Preferred linear volatile silicones that may be used have the general formula:
(CH₃)₃Si—O—[Si(CH₃)₂—O]ₙ—Si—(CH₃)₃
wherein n=0-4.

Examples of such silicones include hexamethyldisiloxane (generally having a viscosity of about 0.65 centipoise), octamethyltrisiloxane (generally having a viscosity of about 1.0 centipoise), decamethyltetrasiloxane (generally having a viscosity of about 1.5 centipoise), dodecamethylpentasiloxane (generally having a viscosity of about 2.0 centipoise), and the like, with all viscosity measurements given for room temperature (25° C.). It is noted that centipoise=centistokes×specific gravity (density). As the density of such linear and cyclic volatile silicones is close to 1, then the values for both centipoises and centistokes will be essentially the same.

Linear and cyclic volatile silicones are available from various commercial sources including Dow Corning Corporation, GE Silicones, Shin-Etsu, Goldschmidt, and Wacker. Examples of suitable Dow Corning volatile silicones are those sold under the tradenames Dow Corning 244, 245, 344, and 200 fluids. Suitable volatile silicones sold by GE Silicones include SF1214, SF1528, SFE839, and the like.

(b). Non-Volatile Silicone Oils

Various non-volatile silicones may be present in the composition. Preferred non-volatile silicones have the general formula:
(CX₃)₃Si—O—[Si(CX₃)₂—O]ₙ—Si(CX₃)₃
wherein n is 5 or greater, preferably from about 5 to 1,000,000; and each X is independently H, phenyl, trimethylsiloxy, fluoro, or C₁₋₁₀ alkoxy. Examples of such silicone oils include those referred to as dimethicone, phenyl trimethicone, diphenyl dimethicone, and the like.

2. Organic Oils

The composition of the invention may also contain one or more organic oils. Suitable organic oils include mono-, di-, or triesters, paraffinic hydrocarbons, and the like.

(a). Paraffinic Hydrocarbons

The composition may contain one or more paraffinic hydrocarbons that may be volatile or non-volatile.

(i). Volatile Paraffinic Hydrocarbons

Suitable volatile paraffinic hydrocarbons include those having straight or branched chains having about 5 to 18 carbon atoms, more preferably about 8-18 carbon atoms. Examples include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of about 70-225, preferably about 160 to 190 and a boiling point range of about 30 to 320, preferably 60-260° C., and a viscosity of less than about 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

(ii). Near Volatile or Non-Volatile Paraffinic Hydrocarbons

It may be desired to include one or more near volatile or non-volatile paraffinic hydrocarbons in the composition. Examples of such hydrocarbons include straight or branched chain hydrocarbons having from 18 to 40 carbon atoms such as heneicosane, docosane, n-octadecane, nonadecane, eicosane, tetracosane, pentacosane, hexacosane, heptacosane, octacosane, dotriacontane, tritriacontane, hexatriacontane, hydrogenated polyisobutene, mineral oil, pentahydrosqualene, squalene, squalane, and so on.

(b). Esters

(i). Monoesters

Suitable monoesters are generally formed by the reaction of a monocarboxylic acid and an aliphatic alcohol that may be substituted with one or more substituents such as hydroxyl, alkyl, or alkoxy groups. Such esters preferably have the formula R—COOH wherein R is a C₁₋₄₅ straight or branched chain, saturated or unsaturated alkyl, alkoxy, C₁₋₃₀ alkoxy alkyl, and the like, any of which such mentioned substituents may be substituted with hydroxyl, C₁₋₃₀ alkyl, or C₁₋₃₀ alkoxy groups. Examples of such monoesters include monoesters of fatty acids having from 6 to 30 carbon atoms, such as stearic acid, malic acid, oleic acid, linoleic acid, behenic acid, palmitic acid, myristic acid, and so on. Further examples of monoesters include isostearyl malate, isopropyl palmitate, stearyl stearate, isopropyl malate, hexyl laurate, cetyl isononanoate, butyl oleate, cetyl palmitate, hexadecyl octanoate, and so on.

(ii). Diesters

Suitable diesters that may be used in the compositions of the invention are the reaction product of a dicarboxylic acid and an aliphatic or aromatic alcohol, or alternatively, the reaction product of a monocarboxylic acid and an aliphatic or aromatic alcohol having at least two hydroxyl groups. The dicarboxylic acid or the alcohol may contain from 2 to 45 carbon atoms, and may be in the straight or branched chain, saturated or unsaturated form. In the case where the ester is formed from a dicarboxylic acid, it may be subsituted with one or more hydroxyl groups. The aliphatic or aromatic alcohol may also contain from 2 to 30 carbon atoms, and may be in the straight or branched chain, saturated, or unsaturated form. The aliphatic or aromatic alcohol may also be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl adipate, disostearyl fumarate, and so on.

(iii). Triesters

Suitable triesters that may be used in the compositions include those that are the reaction product of a tricarboxylic acid and an aliphatic or aromatic alcohol, or the reaction product of a mono- or dicarboxylic acid and an aliphatic alcohol having two, three, or more substituted hydroxyl groups. As with the mono- and diesters mentioned above, either the acid or the alcohol or both may contain from about 2 to 30 carbon atoms, and may be saturated or unsaturated, straight or branched chain, and may be substituted with one or more hydroxyl groups.

Preferably, one or more of the acid or alcohol is a fatty acid or alcohol containing from about 6 to 30, preferably from about 14 to 22 carbon atoms. Examples of triesters include triarachidin, tributyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, triisostearyl citrate, and so on.

D. Particulates

The composition of the invention comprises particulates, which include both pigments and powders. The term “powder” refers to white particulates (such as titanium dioxide) or non-pigmented particulates (such as boron nitride, nylon, etc.), that are used for muting color or, with respect to foundations, used for providing opacity or coverage. Preferred compositions of the invention comprise both pigments and powders. Suggest ranges of pigment are from about 0.001-90%, preferably from about 0.005-85%, more preferably from about 0.01-75% by weight of the total composition. Suggested ranges of powders are from about 0.001-90%, preferably from about 0.005-80%, more preferably from about 0.01-70% by weight of the total composition.

1. Pigments

Suitable pigments include inorganic or organic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments also generally consist of insoluble metallic salts of certified color additives, referred to as the Lakes.

Inorganic pigments include iron oxides such as red, black, yellow and the like; ultramarines, chromium, chromium hydroxide colors, and mixtures thereof.

Pigments used in the composition of the invention may be regular sized, ranging from 35-200, from about 40 to 100 microns, or such pigments may be in the microfine particle size form, where the particle sizes range from about 0.05-34 microns, preferably from about 0.1-25 microns, more preferably from about 1-25 microns, most preferably from about 1-15 microns in diameter.

In one preferred embodiment of the invention the pigments are either inherently hydrophilic or are coated with a substance that causes such pigments to be water soluble or water dispersible, preferably water dispersible. Most preferred is where the pigments present in the composition are also oil dispersible and diffusive. Thus, such pigments may be dispersed in the oil phase of the emulsion while the composition is in the resting state. The dispersion of the pigments in the oil phase of the emulsion provides a composition that preferably does not match the skin because the full color of the pigments is not visible when suspended or dispersed in the oil phase. In one embodiment of the invention, the color of the composition in the resting state when the pigments are suspended in the oil phase may be white, grayish white, whitish blue, or a variety of other colors depending on the color of the powders or other non-diffusive pigments that are used in the composition. However, when the composition is applied to skin, the diffusive pigments diffuse out of the oil phase and into the water phase or onto the keratinous surface, causing the color of the composition to develop directly on the keratinous surface when it is applied. The color development may be attributed to the contact of the pigment with the water in the formula or on the skin, the mechanical agitation generally found in the application process, or the temperature or pH change when the composition is applied to the keratinous surface, or when the composition containing such pigments comes into contact with the oil or water on the skin.

In the case of a composition applied to skin such as foundation makeup, the development of the color directly on the skin from a non-skin matching color to a skin matching color gives the consumer the impression that the composition is “smart” and capable of changing color to exactly match her skin tone.

If the pigments are coated with a substance in order to make them soluble, or dispersible or suspendable, in the oil phase of the emulsion, a variety of materials are suitable including polysaccharides, carbohydrates, or biological polymers. While such treatment materials may, in general, be generally known to be hydrophilic in nature, they may still cause the pigment itself to be dispersible rather than soluble in water.

Examples of biological polymers suitable as pigment coating materials for the pigments used in the compositions of the invention include those having repeating monomers that are generally of the carbohydrate type. Included are materials obtained from edible grains such as corn starch; or chemically modified materials obtained from edible grains. Examples of such biological polymers include, but are not limited to, biosaccharide gum; chitosan and derivatives thereof such as butoxy chitosan, carboxymethyl chitosan, carboxybutyl chitosan, chitosan gluconate, chitosan adipate, chitosan glycolate, chitosan lactate, etc; chitin and derivatives thereof such as carboxymethyl chitin, chitin glycolate; cellulose and derivatives thereof such as cellulose acetate; microcrystalline cellulose; distarch phosphate; sodium hyaluronate; soluble proteoglycan; galactoarabinan; glycosaminoglycans; glycogen; sclerotium gum; dextran; starch and derivatives thereof, and the like.

Examples of carbohydrates suitable for use in coating pigments for use in the compositions of the invention may be described as polyhydroxy aldehydes or ketones that conform to the general structure:
Cₓ(H₂O)_{y}
wherein x and y range from about 1 to 1,000,000. The carbohydrates may be monosaccharides, disaccharides, or polysaccharides. Examples of suitable carbohydrates include those set forth on pages 1660 through 1662 of the International Cosmetic Ingredient Dictionary and Handbook, Volume 2, Eighth Edition, 2000, which is published by The Cosmetic, Toiletry, and Fragrance Association (C.T.F.A.), hereby incorporated by reference in its entirety. Such carbohydrates include, but are not limited to, amylodextrin, betaglucan, cyclodextrin, modified corn starch, glycogen, hyaluronic acid, hydroxypropyl cyclodextrin, lactose, maltitol, guanosine, glyceryl starch, triticum vulgare starch, trehalose, sucrose or derivatives thereof; raffinose, sodium chondroitin sulfate, and the like.

Also suitable for treating the pigments are C₁₋₂₀ alkylene glycols or C₁₋₂₀ alkylene glycol ethers either alone or in combination with tri-C₁₋₂₀ alkylsilanes with the alkyl groups ranging from about C₁₋₂₀. Examples of such pigments include PEG alkyl ether alkoxy silane treated pigments including but not limited to PEG-8 methyl ether triethoxysilane treated pigments sold by Kobo under the brand name “SW” pigments.

Also suitable are pigments treated with silicones such as dimethicones having hydrophilic groups also known as dimethicone copolyols or alkyl dimethicone copolyols. In particular, such dimethicones include those having C₁₋₂₀ alkylene oxide repeating units such as ethylene, propylene, and the like. One particular suitable pigment is PEG-12 dimethicone treated pigment sold by Sensient Corporation under the trade name LCW AQ pigments.

Also suitable are pigments that are treated with isopropyl titanium triisostearate. Such pigments are sold by Kobo Products under the trade name ITT Pigments.

Other suitable pigments are those treated with fluorinated compounds such as fluorinated silicones, fluorinated perfluoroethers, or fluorinated paraffinic hydrocarbons. One example of such pigment is sold by Cardre under the brand name FHC, which are polyperfluoromethylisopropyl ether treated pigments, particularly iron oxides.

Particularly preferred is where the makeup contains iron oxide pigments, which are coated or treated with galactoarabinan, which is a polysaccharide obtained from the extraction of the larch tree Larix. Such pigments are available from Color Techniques under the GA series. For example, suitable iron oxides include GA-7293 hydrophilic red iron oxide, GA-7131 hydrophilic yellow iron oxide, GA-7125 hydrophilic red iron oxide, or GA-7403 hydrophilic black iron oxide, all sold by Color Techniques. Preferred is where from about 10 to 100, preferably from about 20 to 100 parts, more preferably from about 40 to 100 parts by weight of the total pigment present comprises diffusive pigments

2. Powders

The composition also contains powders or fillers that are present for adjusting the color of the composition, and in some cases may provide a sunscreen effect by physical blocking of UV radiation. Preferably, the particle size of the powders ranges from 0.05 to 100 microns, and are present in ranges of 0.001-90%, preferably 0.005-80%, more preferably from about 0.01-70% by weight of the total composition.

Examples of powders include white or non-pigmentitious powders such as titanium dioxide, bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, chalk, hydrated silica, kaolin, magnesium aluminum silicate, magnesium trisilicate, montmorillonite, talc, mica, zinc laurate, zinc myristate, zinc rosinate, nylon, silica silylate, silk powder, sericite, tin oxide, titanium hydroxide, trimagnesium phosphate, walnut shell powder, and the like. While titanium dioxide is commonly considered to be a white pigment when used in paints, in color cosmetic compositions it is used more for its ability to mute color, and/or provide an opaque or semi-opaque finish, or provide sunscreen protection, then as a colorizing ingredient. The above mentioned powders may be surface treated with lecithin, amino acids, mineral oil, silicone oil or various other agents either alone or in combination, which coat the powder surface and render the particles more lipophilic in nature.

Alternatively, the powders may be coated with a substance including those mentioned herein with respect to the pigments. The powders treated with such a substance may then be suspended or dispersed in the oil or water phase of the emulsion, preferably the oil phase.

Most preferred is where the powder component of the composition is dispersed in the oil phase and contains a portion of powders in the microfine particle size form so as to minimize the opacity of the resulting composition and thereby enable the composition to match plurality of skin shades in one skin tone category. Preferred microfine particle sizes range from about 0.1-30 microns, more preferably from about 0.5-20 microns, most preferably from about 1-17 microns in diameter. In the most preferred embodiment of the invention, at least a portion of the titanium dioxide present in the formula will be in the ultrafine form; or more preferably from about 0.1-15%, preferably from about 0.5-10% by weight of the total composition will be microfine particle titanium dioxide; with the remaining amount of titanium dioxide (ranging from about 0.1-5% by weight of the total composition) being of a regular particle size, ranging from about 35-100 microns. It is noted that typical foundation makeup compositions generally contain from about 7 to 15% of titanium dioxide powder, with such titanium dioxide generally being of a standard particle size of about 50 microns. This provides the resulting foundation with an appreciable degree of opacity, which is excellent for covering skin imperfections. In the makeup composition of the invention, the substitution of ultrafine particle size powders for regularly sized particles will provide a composition with substantially reduced opacity. This will provide a makeup that provides a more sheer finish on the skin, which in turn ensures that a single foundation makeup formula will more easily match a plurality of skin tones. The pigments present in the composition will accent the color of the skin, and if they are microfine pigments will tend to do so in a very subtle manner. At the same time, the microfine powders present will mute the pigments to provide the desired skin tone, but will not provide an amount of opacity that will compromise the versatility of the composition in matching a variety of skin tones. Additionally, the microfine powder particles will contribute to the resting color of the composition, so that in the most preferred embodiment the composition appears white, whitish gray, or possibly whitish blue while in the resting state, but upon application to the skin the diffusive pigments diffuse from the oil phase in which they are suspended, and the color of the composition develops to match the skin tone of the individual to which it is applied.

The microfine particle size powders may confer an additional benefit of providing SPF to the composition. For example, very fine particle zinc oxide and/or titanium dioxide may cause the composition to exhibit SPF (sun protective factor) values as high as 5 to 25. For example, if the composition contains from about zinc oxide, titanium dioxide, or mixtures thereof, having particle sizes ranging from about 0.005 to 10 microns, the composition will likely have SPF values ranging from 5 to 25.

Further, it is noted that in general, the powder component (or the portion of powders in the formula) in typical foundation makeup generally ranges from about 5 to 30% by weight of the total foundation composition. In order to provide a foundation that better matches a plurality of skin shades in one skin tone category, a portion of such powders in the powder component must be found in the microfine particle size range (preferably the majority of particles in the 1-17 micron size range). Preferred is where about 35-100%, preferably from about 40-95%, or parts by weight, of the total powder component comprises powders in the microfme particle size range. Most preferred is where the powder component present in the foundation contains from about 45-95% or parts by weight of the total powder component of microfine titanium dioxide as the powder component. It has been found that where at least 35-100% by weight of the total powder component is microfine, or where from about 30-95% by weight of the total titanium dioxide content is in the microfine particle size form, the resulting foundation will match a plurality of skin shades in one skin tone category.

In one alternate embodiment of the invention the powders may be coated with the substance mentioned herein, and dispersed in the oil phase of the emulsion, with the pigments in the composition being dispersed or solubilized in the aqueous phase or the oil phase depending on whether they are hydrophilic or lipophilic in nature. For example, such a composition might contain powders such as titanium dioxide coated with a hydrophilic material and dispersed in the oil phase of the emulsion. The water phase of the emulsion might contain water soluble pigments in the water phase. Depending on the type and amount of pigments in the water phase, the composition may exhibit a certain colored resting state. Then, when such a composition is applied to the keratinous surface, the powder coated with hydrophilic substance will diffuse from the oil phase and cause the composition to lighten, or perhaps become white or lightly tinted in color.

In yet another example, the emulsion composition might contain both powders and pigments suspended in the oil phase. The resting state color of the composition may be clear or translucent. Upon application to the desired keratinous surface, the composition will change color to a tinted colored shade depending on the amount of pigments and powders present.

A variety of other variations are possible depending on the pigments and powders used and whether they are suspended in the oil phase or water phase.

E. Mono-, Di-, or Polyfunctional Organic Alcohols

It may be desirable to include one or more mono-, di-, or polyfunctional organic alcohols in the composition. If present, such alcohols may range from about 0.001-25%, preferably from about 0.005-20%, more preferably from about 0.01-15% by weight of the total composition.

Such alcohols may function as non-aqueous solvents, humectants, astringents, and the like. Preferably, such alcohols have the general formula R—OH, where R is a C₂₋₁₀ straight or branched chain alkyl that may be unsubstituted or substituted with one or more hydroxyl groups; a C₂₋₁₀ alkyl amido alkyl, or a C₂₋₁₀ alkyl amido alkoxy. Preferably R has one or more substituted hydroxyl groups, making R—OH polyhydric. The alcohol may also be a dispersant for one or more ingredients in the formula.

Examples of suitable alcohols include isopropanol; ethanol; alkylene glycols such as ethylene, propylene, or butylene glycol; glycerin; panthenol; panthenyl ethyl ether; phytantriol; or mixtures thereof.

Panthenol is a trihydric alcohol of the formula R—OH wherein R is an alkyl amido alkyl having two substituted hydroxyl groups. It has the formula:

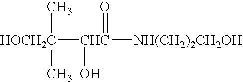

Panthenyl ethyl ether, a monohydric alcohol, is the ethyl ether of panthenol having the formula:

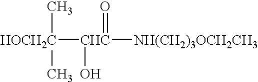

Phytantriol is an aliphatic polyhydric alcohol having the general formula:

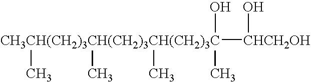

F. Surfactants

Preferably the composition contains one or more surfactants that are present in an amount sufficient to form a stable emulsion. Suggested ranges of surfactant may be from about 0.001-45%, preferably from about 0.005-35%, more preferably from about 0.01-10% by weight of the total composition. Suitable surfactant may be organic or silicone based.

1. Silicone Surfactants

Preferred nonionic silicone surfactants include those having at least one hydrophilic radical and at least one lipophilic radical. These silicone surfactants may be a liquid or solid at room temperature and are water-in-oil or oil-in-water type surfactants that have a Hydrophile/Lipophile Balance (HLB) of about 2 to 18. Preferably the silicone surfactant is a nonionic surfactant having an HLB of about 2 to 12, preferably about 2 to 10, most preferably about 4 to 6. The HLB of a nonionic surfactant is the balance between the hydrophilic and lipophilic portions of the surfactant and is calculated according to the following formula:
HLB=7+11.7×log M_{w}Mₒ
where M_{w} is the molecular weight of the hydrophilic group portion and Mₒ is the molecular weight of the lipophilic group portion.

The polymeric silicone surfactant used in the composition may have any of the following general formulas:
MₓQ_{y}, or
MₓT_{y} or
MDₓD′_{y}D″_{z}M
wherein:

each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof,

T is a trifunctional siloxy unit having the empirical formula R′SiO_{1.5} or RSiO_{1.5} wherein R is methyl and R′ is a C₂₋₂₂ alkyl or phenyl.

Q is a quadrifunctional siloxy unit having the empirical formula SiO_{4/}2; and

D, D′, D″, x, y, and z are as set forth below, with the proviso that the compound contains at least one hydrophilic radical and at least one lipophilic radical. Preferred is a linear silicone of the formula:
MDₓD′_{y}D″_{z}M
wherein M=RRRSiO_{1/2}
D=RRSiO_{2/2}
D′=RR′SiO_{2/2}
D″=R′R′SiO_{2/2}
x, y, and z are each independently 0-1000,
where R is methyl or hydrogen, and R′ is a hydrophilic radical or a lipophilic radical, with the proviso that the compound contains at least one hydrophilic radical and at least one lipophilic radical.

Most preferred is wherein
M=trimethylsiloxy
D=[Si—(CH₃)₂—O]_{2/2}
D′=Si—[(CH₃)][(CH₂)ₙCH₃]O_{2/2} where n=0-40,
D″=Si—[(CH₃)][(CH₂)ₒ—O—PE)]O_{2/2} where PE is (—C₂H₄O)ₐ(—C₃H₆O)_{b}H, o=0-40,
a=1-100 and b=1-100, and

More specifically, suitable silicone surfactants have the formula: 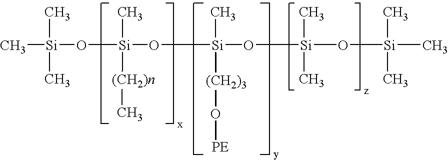
wherein n is 0-40, preferably 12-18, most preferably 14; and
PE is (—C₂H₄O)ₐ(—C₃H₆O)_{b}—H
where x, y, z, a, and b are such that the maximum molecular weight of the polymer is approximately 50,000. An example of such a silicone surfactant is where n=14, having the C.T.F.A. name cetyl dimethicone copolyol. Cetyl dimethicone copolyol may be referred to more specifically by enumerating the number of repeating ethylene oxide and propylene oxide units in the polymer. For example, one type of suitable cetyl dimethicone copolyol for use in the invention may be cetyl PEG/PPG-10/1 dimethicone having 10 PEG units for every 1 PPG unit.

Another type of silicone surfactant that may be used in the compositions of the invention are emulsifiers sold by Union Carbide under the Silwet™ trade mark, which are referred to by the C.T.F.A. name dimethicone copolyol. One type of dimethicone copolyol may be more specifically referred to as PEG/PPG 18/18 dimethicone, which is dimethicone having 18 PEG units and 18 PPG units on the EO (ethylene oxide)/PO (propylene oxide) substituent.

Also suitable as nonionic silicone surfactants are hydroxy-substituted silicones such as dimethiconol, which is defined as a dimethyl silicone substituted with terminal hydroxy groups.

Examples of suitable silicone surfactants are those sold by Dow Corning under the trade name Dow Corning 3225C or 5225C Formulation Aid, Dow Corning 190 Surfactant, Dow Corning 193 Surfactant, Dow Corning Q2-5200, and the like are also suitable. In addition, surfactants sold under the trade name Silwet by Union Carbide are also suitable. Preferred silicone surfactants for use in the compositions of the invention are dimethicone copolyol or cetyl dimethicone copolyol.

2. Organic Surfactants

The composition may contain one or more organic surfactants either in lieu of, or in combination with, the silicone surfactants mentioned above.

(a). Alkoxylated Alcohols or Ethers

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Steareth 2-100, formed by the reaction of stearyl alcohol and ethylene oxide where the number of repeating ethylene oxide units ranges from 2 to 100; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, where the number of repeating ethylene oxide units is 1 to 45; laureth 1-100 formed by the reaction of lauryl alcohol and ethylene oxide where the number of repeating ethylene oxide units is 1 to 100; and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol, are also suitable.

(b). Alkoxylated Carboxylic Acids

Also suitable surfactants are alkyoxylated carboxylic acids, which are formed by the reaction of a carboxylic acid with an alkylene oxide or with a polymeric ether. The resulting products have the general formula: 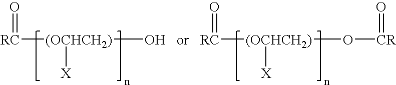
where RCO is the carboxylic ester radical, X is hydrogen or lower C₁₋₄ alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

(c). Monomeric or Polymeric Ethers

Suitable surfactants also include monomeric, homopolymeric or block copolymeric ethers. Such ethers are formed by the polymerization of monomeric alkylene oxides, generally ethylene or propylene oxide. Such polymeric ethers have the following general formula: 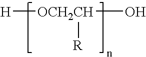
wherein R is H or lower C₁₋₄ alkyl and n is the number of repeating monomer units, and ranges from 1 to 500.

(d). Sorbitan Derivatives

Other suitable nonionic surfactants include derivatives of sorbitan, for example form by the alkoxylation of sorbitan, or by the reaction of C₁₋₂₅, preferably C₆₋₂₀ carboxylic acids with sorbitol or hexitol anhydrides derived from sorbitol.

For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85.

Examples of sorbitan derivatives include the reaction product of sorbitol or the hexitol anhydrides thereof with fatty acids to form derivative such as sorbitan oleate, sorbitan palmitate, sorbitan sesquiisostearate, sorbitan stearate, sorbitan sesquioleate, and so on.

G. Waxes

It may be desirable to include one or more waxes in the composition to increase viscosity, provide stability, or for other functional purposes. If present, suggested ranges of such waxes are from about 0.01-45%, preferably 0.05-35%, more preferably from about 0.1-25% by weight of the total composition. Such waxes may be solid or semi-solid at room temperature. The waxes preferably have a melting point of about 39 to 135° C., preferably in the range of 45 to 95° C., most preferably 55 to 95° C.

Suitable waxes generally include animal waxes, plant waxes, mineral waxes, silicone waxes, synthetic waxes, and petroleum waxes. More specifically, these waxes include tribehenin, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like. Particularly preferred is where the wax is an organic wax, tribehenin.

H. Emulsion Stabilizers

The composition of the invention may contain one or more ingredients that stabilize the emulsion, when the composition is in the emulsion form. If present, suggested ranges of emulsion stabilizers are from about 0.001-10%, preferably from about 0.005-8%, more preferably from about 0.01-3% by weight of the total composition. Suitable emulsion stabilizers include alkali metal or alkaline earth metal salts such as magnesium sulfate, sodium chloride, magnesium chloride and the like; or EDTA, HEDTA or derivatives thereof. Preferred emulsion stablizers include sodium chloride, tetrasodium EDTA, or mixtures thereof.

I. Film Forming Ingredients

If desired, the composition may contain one or more film forming ingredients. If present, the film former may range from about 0.1-45%, preferably about 0.5-20%, more preferably about 1-15% by weight of the total composition. Suitable film formers include resinous plant extracts, synthetic polymers, and the like.

1. Resinous Plant Extracts

Examples of resinous plant extracts that provide film forming properties include materials such as rosin and shellac, or derivative thereof.

2. Synthetic Polymeric Film Formers

Suitable synthetic polymers may be silicone or organic based. Particularly preferred are siloxy silicate polymers having the following general formula: 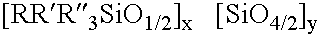
wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1.

Preferably R, R′ and R″ are each a C₁₋₆ alkyl, and more preferably are methyl and x and y are such that the ratio of (CH₃)₃SiO_{1/2} units to SiO_{4/2} units is about 0.75 to 1. Most preferred is this trimethylsiloxy silicate containing 2.4 to 2.9 weight percent hydroxyl groups, which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the trade name 749 Fluid, which is a blend of about 40-60% volatile silicone and 40-60% trimethylsiloxy silicate. Dow Corning 749 Fluid, in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

Also suitable are synthetic polymers that are often found in the form of an aqueous dispersion where the polymer particles are dispersed in the aqueous phase of the polymer emulsion. Examples of such polymers include homo- or copolymers of monomers such as acrylic acid, methacrylic acid or C₁₋₃₀ esters of acrylic or methacrylic acid, vinyl pyrrolidone, vinyl acetate, urethane, C₁₋₃₀ hydroxy esters of acrylic or methacrylic acid, vinyl isodecanoate, styrene, and olefins such as ethylene, propylene, butene, pentene, decene, hexadecene, and so on.

J. Finish Enhancers

The composition may contain one or more compounds that enhance the finish, or aesthetic properties, of the composition after it is applied to the desired keratinous surface. Preferred finish enhancers are synthetic elastomers, which may be silicone elastomers or organic polymers having elastomeric properties. The term “elastomer”means a compound exhibits properties associated with rubber, such as extensibility with applied force, retractibility upon release of the force, and lack of permanent deformation as a result of extension. Rubber like properties are generally seen in high molecular weight cross-linked polymers having weak intermolecular forces. If present, suggested ranges are from about 0.001-20%, preferably from about 0.005-15%, more preferably from about 0.01 -10% by weight of the total composition.

Preferred elastomers are generally in the solid particulate form having particle size ranging from about 0.05 to 75 microns. Elastomers will often provide a velvety smooth finish to the composition, improved spreadability and blendability, and a light, non-greasy feel.

1. Synthetic Organic Polymeric Elastomers

A variety of cross-linked synthetic organic polymeric elastomers may be used as finish enhancers, including those polymerized from various types of ethylenically unsaturated monomers such as acrylic acid, methacrylic acid, and simple esters thereof, vinyl monomers such as vinyl acetate, vinyl isodecanoate, methyl vinyl ether; maleic anhydride. These monomers may be copolymerized with one or more organic compounds such as esters, glycols, fatty acids, and so on. Examples of such polymers include acrylates/VA crosspolymer, acrylates/vinyl isodecanoate crosspolymer, adipic acid/diethylene glcyol/glycerin crosspolymer, allyl methacrylates crosspolymer, HDI/trimethylolhexyllactone crosspolymer, lauryl acrylate/VA crosspolymer, methyl methacrylate crosspolymer, PVM/MA decadiene crosspolymer, PEG crosspolymer, PPG-35/PPG-51 glyceryl ether/IPDI crosspolymer, trimethyl pentanediol/adipic acid/glycerin crosspolymer, and so on. One preferred type of elastomer is HDI/trimethylolhexyllactone crosspolymer which is a crosslinked condensation polymer formed from the reaction of hexyldiisocyanate with the esterification product of trimethylolpropane with 6 to 7 moles of hexyllactone. This polymer is available from Kobo Products under the tradename BPD-500, which is a mixture of silica and the polymer having the C.T.F.A. name HDI/trimethylol hexyllactone crosspolymer. It is a fine white powder having a particle size of about 5-20 microns comprising about 95-99% polymer and 1-5% silica.

2. Silicone Elastomers

Also suitable for use as finish enhancers are silicone elastomers such as those disclosed in U.S. Pat. No. 6,171,581, which is hereby incorporated by reference in its entirety. Examples of such elastomers include cetearyl dimethicone/vinyl dimethicone crosspolymer, dimethicone copolyol crosspolymer, dimethicone crosspolymer, dimethicone/phenyldimethicone crosspolymer, dimethicone/vinyl dimethicone crosspolymer, and mixtures thereof.

K. Botanical Ingredients

The composition may contain one more botanical ingredients that are derived from plants, flowers, herbs, and the like. If present, such botanicals may range from about 0.001-10%, preferably from about 0.005-8%, more preferably from about 0.01-5% by weight of the total composition.

Suitable botanicals include extracts of aloe, gingko biloba, panax ginseng, camellia sinensis, centaurea cyanus flower extract, grape seed extract, and the like.

L. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D, C, and K, as well as derivatives thereof are suitable. Particularly preferred are derivatives of vitamins C, E, and A such as magnesium ascorbyl phosphate, retinyl palmitate, tocopheryl acetate, and mixtures thereof.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

M. Other Ingredients

A variety of other ingredients may be used in the composition, such as fragrances, preservatives, alpha or beta hydroxy acids, or mixtures thereof. A suggested range for the totality of such ingredients is about 0.001 to 3% based on the total weight of the composition. Typical preservatives include methyl, ethyl, and propyl parabens and phenoxyethanol. Suitable alpha or beta hydroxy acids include glycolic, malic, lactic, and salicylic acids.

N. Makeup Shades and Skin Tones

Set forth below is a table identifying certain commercially available foundation makeup brands and shades, and listing shades that are considered to fall within the various skin tone categories listed. It is noted that in some cases the various brands do not sell products in all skin tone categories. For example, in many brands there are no shades that fall into the “dark” category, meaning that the cosmetic manufacturer does not offer shades suitable for consumers who have a skin tone falling in that category.

| | | | Total Powder | Total Titanium |
|---|---|---|---|---|
| Skin Tone | | | Content wt % | Dioxide Content |
| Category | Shade | Brand | (range) | wt % (range) |
| Light | Pale | Almay Amazing | 10-25 | 5-12 |
| | Buff | Lasting Makeup | | |
| | Naked | | | |
| | Neutral | | | |
| Light/medium | Sand | | | |
| Medium | Warm | | | |
| | Honey | | | |
| | Tan | | | |
| Light | Ivory | Almay Clear | 8-26 | 4-11 |
| | Naked | Complexion Blemish | | |
| | Neutral | Healing Makeup | | |
| Light/Medium | Sand | | | |
| | Bisque | | | |
| | Beige | | | |
| Medium | Warm | | | |
| | Golden | | | |
| | Honey | | | |
| Light | Porcelain Beige | Almay Time-off | 5-15 | 3-12 |
| | Ivory Beige | Age Smoothing | | |
| | Natural Beige | Makeup | | |
| Light/medium | Soft Beige | | | |
| | Cream Beige | | | |
| Medium | Honey Beige | | | |
| | Almond Beige | | | |
| | True Beige | | | |
| Light | Nearly Nude | Almay Nearly | 5-18 | 2-11 |
| | Nearly Neutral | Naked Touch-Pad | | |
| Light/medium | Nearly Sand | Liquid Makeup | | |
| | Nearly Beige | | | |
| Medium | Nearly Warm | | | |
| | Nearly Honey | | | |
| Light | Pale | Almay Kinetin | 8-30 | 5-15 |
| | Buff | Skin-Smoothing | | |
| | Naked | Foundation | | |
| Light/Medium | Neutral | | | |
| | Sand | | | |
| | Warm | | | |
| Medium | Honey | | | |
| | Tan | | | |
| | Caramel | | | |
| Light | Pale | Almay Wake-Up | 5-25 | 5-18 |
| | Buff | Call! | | |
| | Naked | Energizing Makeup | | |
| Light/medium | Neutral | | | |
| | Sand | | | |
| | Warm | | | |
| Medium | Honey | | | |
| | Soft Tan | | | |
| | Tan | | | |
| | Caramel | | | |
| Dark | Mocha | | | |
| | Expresso | | | |
| Light | Ivory | Revlon Colorstay | 8-30 | 4-15 |
| | Buff | Stay Natural | | |
| | Nude | Makeup | | |
| Light/medium | Sand Beige | | | |
| | Natural Beige | | | |
| | Medium Beige | | | |
| Medium | Honey Beige | | | |
| | True Beige | | | |
| | Natural Tan | | | |
| Dark | Toast | | | |
| | Caramel | | | |
| | Cappuccino | | | |
| Light | Ivory | Revlon Colorstay | 5-30 | 3-16 |
| | Buff | Makeup | | |
| | Nude | | | |
| Light/Medium | Sand Beige | | | |
| | Natural Beige | | | |
| | Medium Beige | | | |
| Medium | True Beige | | | |
| | Toast | | | |
| | Tawny | | | |
| | Natural Tan | | | |
| Dark | Early Tan | | | |
| | Rich Tan | | | |
| | Caramel | | | |
| | Cappuccino | | | |
| | Mahogany | | | |
| | Mocha | | | |
| Light | Ivory Beige | Revlon Age Defying | 7-29 | 3-18 |
| | Nude Beige | Makeup and Concealer | | |
| Light/Medium | Sand Beige | Compact with Botafirm | | |
| | Natural Beige | | | |
| | Medium Beige | | | |
| Medium | Honey Beige | | | |
| | Natural Tan | | | |
| | Early Tan | | | |
| Light | Fresh Ivory | Revlon Age-Defying | 7-30 | 3-19 |
| | Bare Buff | Makeup with Botafirm | | |
| | Nude Beige | for Dry Skin | | |
| | Soft Beige | | | |
| Light/Medium | Natural Beige | | | |
| | Medium Beige | | | |
| | Cool Beige | | | |
| | Sand Beige | | | |
| Medium | Honey Beige | | | |
| | Golden Beige | | | |
| | Early Tan | | | |
| | Rich Tan | | | |
| Light | Fresh Ivory | Revlon Age Defying | 7-28 | 3-17 |
| | Bare Buff | Makeup with Botafirm | | |
| | Nude Beige | for Normal/ | | |
| | Soft Beige | Combination Skin | | |
| Light/Medium | Natural Beige | | | |
| | Medium Beige | | | |
| | Cool Beige | | | |
| | Soft Beige | | | |
| Medium | Honey Beige | | | |
| | Golden Beige | | | |
| | Early Tan | | | |
| | Rich Tan | | | |
| Light | Fair | Revlon Age Defying | 3-25 | 2-14 |
| | Light | Light Makeup with | | |
| Light/Medium | Light Medium | Botafirm for Every | | |
| Medium | Medium | Skin | | |
| | Medium Deep | | | |
| Light | Ivory Beige | Revlon New | 5-28 | 3-17 |
| | Creamy Peach | Complexion Oil-Free | | |
| | Beige | Liquid Makeup | | |
| | Cameo Beige | | | |
| Light/Medium | Natural Beige | | | |
| | Warm Beige | | | |
| | Sun Beige | | | |
| Medium | Natural Rose | | | |
| | Beige | | | |
| | Sand Beige | | | |
| | Medium Beige | | | |
| Light | Ivory Beige | Revlon New | 6-30 | 2-19 |
| | Tender Peach | Complexion One-Step | | |
| Light/Medium | Sand Beige | Compact Makeup | | |
| | Natural Beige | | | |
| | Medium Beige | | | |
| Medium | Honey Beige | | | |
| | Warm Beige | | | |
| | Toast | | | |
| | Natural Tan | | | |
| Dark | Rich Tan | | | |
| | Caramel | | | |
| | Cool Beige | | | |
| Light | Buff Beige | L'Oréal True Match ® | 5-30 | 2-14 |
| | Classic Ivory | Liquid Makeup | (estimated) | (estimated) |
| | Natural Ivory | | | |
| | Natural Buff | | | |
| Light/Medium | Sand Beige | | | |
| | Nude Beige | | | |
| | Natural Beige | | | |
| | Creamy Natural | | | |
| Medium | True Beige | | | |
| | Sun Beige | | | |
| | Buff Beige | | | |

The above commercial foundation products contain a powder component that includes titanium dioxide. Many of the brands noted exhibit a multiplicity of different shades for each tone category. If microfine particles are used for a portion of the powder component, more preferably if microfine titanium dioxide particles are used for a portion of the titanium dioxide component, in accordance with the invention, the resulting foundation will have a more universal character, e.g. will match to a wider variety of skin shades in one skin tone category.

It is further noted that the composition and method of the invention may also be used with products such as blush on, concealer, eye shadow, mascara, eyeliner, lipstick, bronzer, and the like. For example, there is a commercial advantage in providing formulas for blush where only a few blush colors may be offered and those colors are more universal amoung a wide variety of skin shades. Rather than selling 15-20 different SKUs of blush, all having a different shade, the cosmetics manufacturer may use the technology of the invention to provide from 1 to 5 shades that may be used across all different skin shades in the various skin tone categories.

Similarly, typically cosmetics manufacturers offer concealers in light, medium, or dark shades. The compositions and methods of the invention will enable the cosmetics manufacturer to even reduce the number of concealer offerings to one or two.

The compositions and methods of the invention may also be used for lipcolor, eyeshadow, and a variety of other cosmetic products where it is desired to reduce the number of SKUs by providing more a more universal color suitable for skin shades across a variety of skin tone categories; and/or where it is desirable to have the cosmetic composition develop color on the skin when applied in order to provide a composition that appears “smart”.

The technology may also be used for products applied to keratinous surfaces such as eyelashes, eyebrows, or nails. Such products include mascara, eyeliner, brow color, nail enamel, and so on.

The invention will be further described in connection with the following examples, which are set forth for purposes of illustration only.

### EXAMPLE 1

A base composition (“A” ) was prepared as follows:

| | | % by |
|---|---|---|
| Seq. | Ingredient | weight |
| 1 | Cyclopentasiloxane/PEG/PPG-18/18 Dimethicone | 16.40 |
| 2 | Cyclohexasiloxane, cyclopentasiloxane | 1.01 |
| 3 | Cyclomethicone, trimethylsiloxysilicate | 1.51 |
| 4 | Sorbitan sesquioleate | 0.05 |
| 5 | Propyl paraben | 0.10 |
| 6 | Titanium dioxide, alumina, methicone | 5.05 |
| 7 | Titanium dioxide, methicone | 2.02 |
| 8 | Zinc oxide, methicone | 2.22 |
| 9 | Talc, methicone | 2.73 |
| 10 | Nylon-12 | 2.02 |
| 11 | Alumina | 0.30 |
| 12 | Boron nitride | 0.71 |
| 13 | HDI/trimethylol hexyllactone crosspolymer, silica | 2.17 |
| 14 | Acrylonitrile/methacrylonitrile/methyl methacrylate | 0.05 |
| | copolymer, iron oxides, talc, water | |
| 15 | Dimethicone | 2.02 |
| 16 | Cetyl PEG/PPG-10/1 dimethicone | 2.02 |
| 17 | Cyclohexasiloxane, cyclopentasiloxane | 10.34 |
| 18 | Tribehenin | 0.05 |
| 19 | Water | 32.68 |
| 20 | Sodium chloride | 1.01 |
| 21 | Tetrasodium EDTA | 0.01 |
| 22 | Aloe barbadensis leaf juice | 0.05 |
| 23 | Sodium hyaluronate, hydrolyzed glycosaminoglycans | 0.20 |
| 24 | Butylene glycol | 3.03 |
| 25 | Methyl paraben | 0.20 |
| 26 | Ethyl paraben | 0.15 |
| 27 | Water, glycerin, PVP, moringa pterygosperma seed | 0.25 |
| | extract | |
| 28-31 | Cyclopentasiloxane, disteardimonim hectorite, | 6.05 |
| | denatured alcohol | |
| 32 | Dimethicone, cyclomethicone, dimethicone/ | 1.51 |
| | cyclomethicone copolymer, ammonium | |
| | polyacryloyldimethyltaurate, polysorbate 20, | |
| | polysorbate 80, tocopheryl acetate | |
| 33 | Dimethicone, trisiloxane | 3.03 |
| 34 | Tocopheryl acetate | 0.05 |
| 35 | Retinyl palmitate | 0.05 |
| 36 | Cyclopentasiloxane, gingoko biloba leaf | 0.25 |
| | extract, panax ginseng root extract, | |
| | camellia sinensis leaf extract, | |
| | centaurea cyanus flower extract, | |
| | vitis vinifera seed extract | |
| 37 | Phenoxyethanol | 0.71 |

The composition was a white liquid.

Three makeup formulas were prepared as follows:

| Ingredient | Light | Light/Medium | Medium |
|---|---|---|---|
| Composition A | 99.1 | 98.7 | 98.5 |
| Iron oxide mixture (light), | 0.9 | — | — |
| galactoarabinan (98:2)¹ | | | |
| Iron oxide mixture (light/ | — | 1.3 | — |
| medium), galactoarabinan (98:2)² | | | |
| Iron oxide mixture (medium), | — | — | 1.5 |
| galactoarabinan (98:2)³ | | | |

¹Iron oxide mixture (light): 81.5 parts yellow iron oxide, 9.50 parts red iron oxide, 9.00 parts black iron oxide.
²Iron oxide mixture (light/medium): 81 parts yellow iron oxide, 11 parts red iron oxide, 8 parts black iron oxide.
³Iron oxide mixture (medium): 65 parts yellow iron oxide, 20 parts red iron oxide, 15 parts black iron oxide.

The makeup formulas were prepared by combining the pre-emulsified Composition A with the pigment blends and mixing well. The pigment blends suspended in the oil phase of the water in oil emulsion. The three compositions were whitish/gray liquids, essentially the same in color. Each of the compositions was stored in a plastic squeeze tube.

When the compositions were squeezed from the tube containers, they were whitish/gray liquids, but after application to the skin and upon rubbing into the skin surface, they formed a color that blended well with the underlying skin color. The composition designated “light”, provided a sheer, color matched finish on a variety of facial skin tones in the light category that typically wear foundation shades referred to as fair, ivory, buff, or nude. The composition designated “light/medium” provided a sheer color matched finish on a variety of facial skin tones in the light/medium category that typically wear foundation shades referred to as sand, natural beige, or medium beige. The composition designated “medium” provided a sheer color matched finish on a variety of facial skin tones in the medium category that typically wear foundation shades referred to as honey beige, warm beige, or early tan. In each case, when the composition matched the skin and provided a light, sheer finish.

### EXAMPLE 2

A composition suitable for use as a mascara or eyeliner is prepared as follows:

| Ingredient | % by weight |
|---|---|
| Water | QS |
| Triethanolamine | 2.25 |
| Acacia Senegal gum | 3.25 |
| Methyl paraben | 0.35 |
| Hydroxyethylcellulose | 0.20 |
| Nylon-12 | 0.10 |
| Magnesium Ascorbyl Phosphate | 0.05 |
| Sorbic acid | 0.20 |
| Panthenol | 0.50 |
| Iron oxides | 0.05 |
| Iron oxides | 0.06 |
| Iron oxides | 0.03 |
| Lecithin, polysorbate 20, sorbitan laurate, propylene | 0.20 |
| glycol stearate, propylene glycol laurate | |
| Simethicone | 0.20 |
| Hydrolyzed corn starch | 0.10 |
| Paraffin | 9.25 |
| Beeswax | 2.78 |
| Stearic acid | 5.55 |
| Glyceryl stearate | 2.31 |
| Propyl paraben | 0.25 |
| Tricontanyl PVP | 1.39 |
| Tocopheryl acetate | 0.20 |
| Hydrogenated stearyl olive ester | 0.25 |
| PPG-17/IPDI/CMPA copolymer | 11.50 |
| Cyclopentasiloxane, dimethiconol | 2.94 |
| Mica, iron oxides, titanium dioxide | 0.01 |
| Retinyl palmitate | 0.01 |
| Diazolidinyl urea | 0.25 |

The above composition is prepared by separately combining the oil and water phase ingredients, then emulsifying them together. After the emulsion is formed, 99 parts of the emulsion and 1 part of galactoarabinan treated black iron oxide pigment mixture are mixed together. The treated pigments are suspended in the oil phase of the emulsion and form a mascara composition that is bluish/black. When applied to the lashes the mascara will form a jet black color on the lashes.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition comprising a first resting color and a second application color, comprising a water phase, and oil phase, and diffusive pigments suspended in the oil phase, wherein the diffusive pigments suspended in the oil phase provide the first resting color and upon diffusion out of the oil phase upon application provide the second application color.

2. The composition of claim 1 wherein the diffusive pigments are organic pigments, inorganic pigments, or mixtures thereof.

3. The composition of claim 1 wherein the diffusive pigments are inorganic pigments.

4. The composition of claim 1 wherein the diffusive pigments are iron oxides.

5. The composition of claim 4 wherein the iron oxides are black, yellow, red, or mixtures thereof.

6. The composition of claim 1 wherein the diffusive pigment is coated with a polysaccharide, carbohydrate, or biological polymer.

7. The composition of claim 1 wherein the diffusive pigment is coated with a saccharide,

8. The composition of claim 1 wherein the diffusive pigment is coated with a mono-, di-, or polysaccharide.

9. The composition of claim 1 wherein the diffusive pigment is coated with a polysaccharide obtained from the extraction of the larch tree.

10. The composition of claim 1 wherein the diffusive pigment is coated with galactoarabinan.

11. The composition of claim 1 in the water and oil emulsion form.

12. The composition of claim 1 in the water in oil emulsion form.

13. The composition of claim 1 wherein the oil phase comprises silicone oil.

14. The composition of claim 1 wherein in the form of water in silicone oil emulsion.

15. The composition of claim 1 in the form of a water in silicone oil emulsion wherein the silicone oils are selected from volatile silicone, non-volatile silicone, or mixtures thereof.

16. The composition of claim 1 in the form of a water in silicone oil emulsion wherein the silicones are selected from volatile silicone, non-volatile silicone, or mixtures thereof; and the composition further comprises at least one nonionic surfactant in an amount sufficient to form an emulsion.

17. The composition of claim 16 wherein the nonionic surfactant is a silicone surfactant.

18. The composition of claim 17 wherein the silicone surfactant is dimethicone copolyol, cetyl dimethicone copolyol or mixtures thereof.

19. The composition of claim 1, which is a foundation makeup comprising, by weight of the total composition, from about:
0.1-99% water,
0.1-95% oil,
0.001-90% pigments,
0.001-90% powders, and
0.001-45% surfactant;
wherein at least about 20 to 100 parts by weight of the total pigment present comprises diffisive pigments and wherein about 30 to 100 parts by weight of the total powder present comprises microfine particle size powders.

20. The composition of claim 19 which is a water and oil emulsion.

21. The composition of claim 19 wherein the surfactant comprises a silicone surfactant.

22. The composition of claim 21 wherein the silicone surfactant comprises dimethicone copolyol, cetyl dimethicone copolyol or mixtures thereof.

23. The composition of claim 19 wherein the oil comprises silicone oil.

24. The composition of claim 23 wherein the silicone oil comprises volatile silicone oil, non-volatile silicone oil, or mixtures thereof.

25. The composition of claim 19 which is a foundation makeup, blush, concealer, mascara, eye shadow, brow color, lipstick, or eye liner.

26. The composition of claim 19 which is a foundation makeup that matches more than one skin shade in one skin tone category.

27. The composition of claim 1 wherein the first resting color is white or whitish gray.

28. The composition of claim 1 wherein the second application color is a flesh color.

29. The composition of claim 1 wherein the composition comprises a foundation make up, blush, concealer, eye liner, eye shadow, mascara, bronzer, or brow color.

30. The composition of claim 1 wherein the diffusive pigment is coated with a material that promotes color change upon the mechanical agitation that occurs in the application process.

31. A cosmetic composition comprising a pigment component and a powder component, the improvement wherein the powder component contains microfine particle size powders in an amount sufficient to provide a composition that will match more than one skin shade in one skin tone category.

32. The composition of claim 31 wherein the pigment component comprises organic pigments, inorganic pigments, or mixtures thereof.

33. The composition of claim 32 wherein the pigment component comprises inorganic pigments.

34. The composition of claim 33 wherein the inorganic pigments are iron oxides.

35. The composition of claim 34 wherein the iron oxides have a particle size ranging from about 0.01-30 microns.

36. The composition of claim 34 wherein the iron oxides are red, yellow, black, or mixtures thereof.

37. The composition of claim 34 wherein the iron oxides are coated with an ingredient that makes the iron oxides hydrophilic.

38. The composition of claim 31 wherein the pigment comprises iron oxides coated with an ingredient that causes such pigment to be suspended in the oil phase of the emulsion when the composition is in the resting state.

39. The composition of claim 38 wherein the iron oxides are coated with one or more mono-, di-, or polysaccharides.

40. The composition of claim 38 wherein the polysaccharide comprises galactoarabinan.

41. The composition of claim 39 wherein the iron oxides are present ranging from about 0.01-75% by weight of the total composition.

42. The composition of claim 31 wherein the powder component comprises titanium dioxide.

43. The composition of claim 42 wherein a portion of the titanium dioxide has a particle size ranging from about 0.01-20 microns.

44. The composition of claim 43 wherein the microfine titanium dioxide is present in the composition in an amount ranging from about 0.5-15% by weight of the total composition.

45. The composition of claim 44 wherein the microfine titanium dioxide present in the composition comprises from about 50 to 95% of the total titanium dioxide content of the composition.

46. The composition of claim 31 wherein the emulsion is a water and silicone oil emulsion and the pigment component comprises diffusive iron oxide pigments suspended in the oil phase of the emulsion.

47. The composition of claim 46 wherein the powder component comprises from about 0.01-70% by weight of the total composition, and wherein from about 50-95% of the total powder component comprises microfine powder particles having a particle size ranging from about 0.1-30 microns.

48. The composition of claim 47 where the composition has a first resting color and a second application color.

49. An emulsion makeup composition having a first resting color and a second application color; comprising a water phase, an oil phase, and diffusive water soluble pigments suspended in the oil phase; wherein the diffusive water soluble pigments suspended in the oil phase provide the first resting color, and upon diffusion out of the oil phase upon application provide the second application color.

50. The composition of claim 49 wherein the first resting color is non-skin-matching.

51. The composition of claim 49 wherein the non-skin-matching color is white or gray.

52. The composition of claim 49 wherein the second application color is skin-matching.

53. The composition of claim 52 wherein the skin matching color is a light, medium, or dark beige skin tone.

53. The composition of claim 49 wherein the diffusive water soluble pigments have a particle size ranging from about 0.001-20 microns.

54. The composition of claim 53 wherein the diffusive pigments are iron oxides.

55. The composition of claim 53 wherein the diffusive pigments are coated with a material that facilitates their suspension or dispersion in the oil phase of the emulsion.

56. The composition of claim 55 wherein the material is a polymer.

57. The composition of claim 56 polymer is a polysaccharide.

59. A water and oil emulsion color cosmetic composition comprising a water phase and an oil phase having diffusive iron oxide pigments suspended therein.

60. The composition of claim 59, which is a foundation, blush, concealer, lipcolor, mascara, eye liner, eye shadow, lipstick, bronzer, or lip liner.
